(19) United States
(12) Patent Application Publication
(10) Pub. No.: US 2022/0080262 A1
(43) Pub. Date: Mar. 17, 2022

(54) METHOD AND APPARATUS TO GENERATE MOTION DATA OF A BARBELL AND TO PROCESS THE GENERATED MOTION DATA

(71) Applicant: Train121 Inc., Clayton, CA (US)

(72) Inventors: Thomas Pluschkell, Clayton, CA (US); Wilson Ruotolo, Palo Alto, CA (US); Franklin Tarke, Sutter, CA (US)

(73) Assignee: Train121 Inc., Clayton, CA (US)

(21) Appl. No.: 17/465,264

(22) Filed: Sep. 2, 2021

Related U.S. Application Data
(60) Provisional application No. 63/077,820, filed on Sep. 14, 2020.

(51) Int. Cl. A63B 24/00; A63B 21/072; G16H 20/30; G06N 3/08

(52) U.S. Cl. CPC A63B 24/0062; A63B 21/0724; G16H 20/30; A63B 2220/17; A63B 2220/40; A63B 2220/833; A63B 2024/0071; G06N 3/08

(57) ABSTRACT

A sensor is coupled to a barbell and generates data indicating motion of a barbell over time. At least one trained neural network is implemented to detect and count repetitions of an exercise performed with the barbell. The at least one trained neural network detects the repetitions based on the data generated by the sensor, and based on (a) a type of exercise performed with the barbell detected from the received data or provided as labeled data, and/or (b) an identity of a user that performed the exercise as detected from the received data or provided as labeled data.

### CROSS-REFERENCE TO RELATED APPLICATIONS

This application claims the benefit of U.S. provisional application Ser. No. 63/077,820, filed Sep. 14, 2020, which is incorporated herein by reference in its entirety.

### BACKGROUND

### 1. Field

This application is related to the use of a sensor coupled to a barbell to generate motion data indicating motion of the barbell, and to processing the generated motion data to make various determinations.

### 2. Description of the Related Art

Conventional Inertial Measurement Unit (IMU) sensors are available that couple to a barbell and generate data indicating motion of the barbell as an exercise is being performed with the barbell. Some conventional IMU sensors use only an accelerometer to generate the data, some conventional IMU sensors use an accelerometer and a gyroscope to generate the data, and some conventional IMU sensors use an accelerometer, a gyroscope, and a magnetometer to generate the data.

Moreover, conventional data analysis techniques typically apply traditional signal processing algorithms to the data generated by the sensor to detect and count repetitions of an exercise performed with the barbell. Such conventional data analysis techniques often apply different signal processing algorithms to the data generated by the sensor for different types of exercises, respectively. For example, a bench press and a squat are different types of exercises that are performed with a barbell. Conventionally, a specific signal processing algorithm may be applied to detect and count repetitions of a bench press, and a different specific signal processing algorithm may be applied to detect and count repetitions of a squat.

Such conventional data analysis techniques are limited in that they require a great understanding of the correspondence of the data generated by the sensor to a given repetition for a type of exercise, so that the signal processing algorithm can be appropriately designed and applied.

Further, in a typical conventional data analysis technique, the type of exercise to be performed must be known so that the appropriate signal processing algorithm for that type of exercise can be applied, and all users are required to strictly adhere to a particular manner of executing a repetition for a specific exercise, which can be difficult and complex in practice. The type of exercise to be performed must either be manually entered into a computer program by a user or automatically generated by a software program so that the user can be informed of what exercise to perform. As a result, users are constrained to follow a pre-assigned exercise routine or interact with a software program, both of which may complicate or distract from the workout.

However, emerging applications have explored the use of machine learning techniques, including Human Activity Recognition (HAR), to associate data generated by the sensor coupled to the barbell to a specific exercise, so that the appropriate signal processing algorithm may be applied to detect and count repetitions. Although this technique allows a user more flexibility in which exercise to complete, such an approach still relies on traditional signal processing algorithms and will incur problems such as those described above.

Further, conventional data analysis techniques typically require a sensor to be assigned to a specific user or group of users in order to correlate the sensor data to the specific user or the specific group of users and their associated program. Again, this can add complexity to the overall process.

As should be understood from the above, there is a need for improved data analysis techniques and programming software that require less user interaction, accommodate more variation on what exercises may be performed and how to perform the exercises, and better detect and count repetitions using barbell motion data.

Moreover, conventional data analysis techniques use the data generated by the sensor to compute various performance metrics. However, there is a need for new personalized performance metrics that can provide additional insights, and for new performance metrics that are “learned” through machine learning techniques.

### BRIEF DESCRIPTION OF THE DRAWINGS

FIG. 1 is a diagram illustrating a sensor and at least one trained neural network, according to an embodiment.

FIGS. 2A and 2B are diagrams illustrating a sensor housed in a housing, as a sensorized collar, according to an embodiment.

FIG. 3 is a diagram illustrating sample data from a prototype sensor implemented as a 9-axis IMU sensor, according to an embodiment.

FIG. 4 is a diagram illustrating an example architecture of a system implementing a sensor and neural networks, according to an embodiment.

FIG. 5 is a diagram illustrating a process of detecting repetitions of an exercise performed with a barbell, from data generated by a sensor coupled to the barbell, according to an embodiment.

FIG. 6 is a diagram illustrating a process of detecting and counting repetitions of an exercise performed with a barbell, according to an additional embodiment.

FIG. 7 is a diagram illustrating the determination of performance metric referred to herein as an intensity index and that can be used for performance assessment, according to an embodiment.

FIG. 8 is a diagram illustrating the determination of a performance metric which may be referred to herein as a silk score and that can be used for performance assessment, according to an embodiment.

FIG. 9 is a diagram illustrating the determination of a performance metric referred to herein as a form factor and that can be used for performance assessment, according to an embodiment.

FIG. 10 is a diagram illustrating the determination of a performance metric referred to herein as a force factor and that can be used for performance assessment, according to an embodiment.

FIG. 11 is a diagram illustrating a performance metric referred to herein as normalized rate of force development (RFD) score and which can be used for performance assessment, according to an embodiment.

FIG. 12 is a diagram illustrating a generalized system architecture and a detailed system architecture, according to an embodiment.

FIG. 13 is a diagram illustrating a system of data aggregation, mapping, and recommendation, according to an embodiment.

FIG. 14 is a diagram illustrating a general configuration of devices that communicate through a network, according to an embodiment.

FIGS. 15A and 15B illustrate a plot of an example multiple input single output recurrent neural network model, according to an embodiment.

### DETAIL DESCRIPTION OF EMBODIMENTS

Reference will now be made in detail to embodiments, examples of which are illustrated in the accompanying drawings. In this regard, the embodiments may have different forms and should not be construed as being limited to the descriptions set forth herein. In order to further clearly describe features of the embodiments, descriptions of other features that are well known to one of ordinary skill in the art may be omitted.

The singular words such as “a,” “an,” and “the”, as used herein, are intended to include the plural forms as well, unless the context clearly dictates otherwise.

The terms “includes,” “comprises,” “including,” and/or “comprising”, when used herein, specify the presence of stated features, figures, steps, components, or combination thereof, but do not preclude the presence or addition of one or more other features, figures, steps, components, or combinations thereof.

Example embodiments may be described with reference to acts and symbolic representations of operations (e.g., in the form of flow charts, flow diagrams, data flow diagrams, structure diagrams, block diagrams, etc.) that may be implemented in conjunction with components, units and/or devices disclosed in more detail below. Although disclosed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

As used herein, the term “and/or” includes any and all combinations of one or more of the associated listed items.

Terminology such as “at least one of A and B”, as used herein, includes any of the following: A, B, A and B.

Terminology such as “at least one of A, B, and C”, as used herein, includes any of the following: A, B, C, A and B, A and C, B and C, A and B and C.

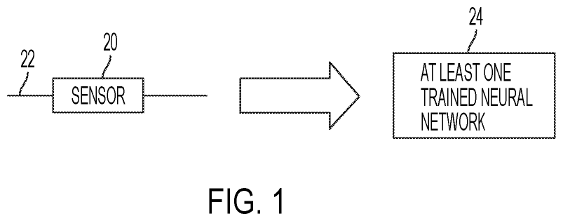

FIG. 1 is a diagram illustrating a sensor and at least one trained neural network, according to an embodiment.

Referring to FIG. 1, a sensor 20 is coupled to a barbell 22. For example, the sensor 20 may be included in a sensorized collar that is coupled to the barbell 22, to thereby couple the sensor 20 to the barbell 22. In other embodiments, the sensor 20 may have a clamp, strap, or other coupling device that may be used to couple the sensor 20 to the barbell 22. However, embodiments are not limited to any particular manner of coupling the sensor 20 to the barbell 22.

Moreover, there are many different barbell shapes and configurations, and embodiments are not limited to any particular barbell shape or configuration.

The sensor 20 generates data indicating at least one of acceleration of the barbell over time, angular velocity of the barbell over time, and magnetic field effects due to movement of the barbell over time.

For example, in an embodiment, the sensor 20 may be a sensor that generates data indicating acceleration of the barbell over time, but does not generate data indicating angular velocity of the barbell over time or data indicating magnetic field effects due to movement of the barbell over time. In another embodiment, the sensor 20 may be a sensor that generates data indicating angular velocity of the barbell over time, but does not generate data indicating acceleration of the barbell over time or data indicating magnetic field effects due to movement of the barbell over time. In another embodiment, the sensor 20 may be a sensor that generates data indicating magnetic field effects due to movement of the barbell over time, but does not generate data indicating of acceleration of the barbell over time or data indicating angular velocity of the barbell over time. In another embodiment, the sensor 20 may be a sensor that generates data indicating acceleration of the barbell over time and data indicating angular velocity of the barbell over time, but does not generate data indicating magnetic field effects due to movement of the barbell over time. In another embodiment, the sensor 20 may be a sensor that generates data indicating acceleration of the barbell over time and data indicating magnetic field effects due to movement of the barbell over time, but does not generate data indicating angular velocity of the barbell over time. In another embodiment, the sensor 20 may be a sensor that generates data indicating angular velocity of the barbell over time and data indicating magnetic field effects due to movement of the barbell over time, but does not generate data indicating acceleration of the barbell over time. In an embodiment, the sensor 20 may be a sensor that generates data indicating acceleration of the barbell over time, data indicating angular velocity of the barbell over time, and data indicating magnetic field effects due to movement of the barbell over time.

Referring again to FIG. 1, the data generated by the sensor 20 may be passed through at least one trained neural network 24 to detect repetitions (which may be referred to herein as “reps”) of an exercise performed with the barbell 22. More specifically, the least one trained neural network 24 may have been trained with labeled data indicating the occurrence of repetitions of an exercise performed with the barbell. Then, the data generated by the sensor 20 is passed through the at least one trained neural network 24, and the at least one trained neural network 24 detects repetitions of an exercise performed with the barbell 22 from the data generated by the sensor 20. In such an embodiment, the at least one trained neural network 24 does not need to know the type of exercise performed with the barbell, or the identity of the user that performed the exercise with the barbell. Therefore, such an embodiment can provide significant advantages over conventional techniques.

In an additional embodiment, prior to detecting the repetitions, the data generated by the sensor 20 may be passed through at least one trained neural network of the at least one trained neural network 24 to detect, from the data generated by the sensor 20, a bounded set of repetitions of the exercise performed with the barbell 22. More specifically, one or more trained neural network of the least one trained neural network 24 may have been trained with labeled data indicating either true sets (i.e., the data corresponds to a type of exercise being performed with a barbell) or false sets (i.e., the data does not correspond to a type of exercise being performed with a barbell). Then, data generated by the sensor 20 is passed through the one or more trained neural network to detect a bounded set. Data of a detected bounded set may then be passed through other trained neural network(s) of the at least one trained neural network 24 to detect repetitions of the exercise performed with the barbell 22 from the data generated by the sensor 20. By detecting a bounded set prior to detecting repetitions, the processing of data is more efficient and, in some cases, more accurate, because data pertaining to extraneous movement (not specific to executing an exercise) may be prevented from being processed by the subsequent neural network(s).

In an additional embodiment, at least one trained neural network of the at least one trained neural network 24 may have been trained using labeled data indicating the total number of repetitions which are contained within a bounded set. In such an embodiment, the data generated by the sensor 20 may be passed through the at least one trained neural network 24, and the at least one neural network 24 determines the number of repetitions of an exercise performed with the barbell 22 within a bounded set of data generated by the sensor 20. In other words, in such embodiments, the at least one neural network 24 can count the number of repetitions, instead of first detecting repetitions which are subsequently counted.

In embodiments disclosed above, no knowledge of the type of exercised performed with the barbell 22 or the identity of the user (that is, a human) that performed with exercise with the barbell 22 is necessary to detect repetitions. Therefore, such embodiments can provide significant advantages over conventional data analysis techniques.

Moreover, such embodiments, which use at least one neural network to detect repetitions, or to detect and count repetitions, provide significant technical benefits over conventional signal processing algorithms that do not use neural networks.

However, in other embodiments, the type of exercise performed with the barbell 22 and the identity of the user that performed the exercise with the barbell 22 may be used to detect repetitions from the data generated by the sensor 20. For example, in an embodiment, the at least one trained neural network 24 may be implemented to (a) detect a type of the exercise performed with the barbell 22 from the data generated by the sensor 20, (b) detect an identity of the user that performed the exercise with the barbell 22 from the data generated by the sensor 20, and (c) detect, or detect and count, the repetitions using the detected type of exercise, the detected identity of the user, and the data generated by the sensor 20. In some embodiments, prior to detecting the type of exercise and the identity of the user, a bounded set of repetitions of the exercise performed with the barbell 22 may be detected. Data from the bounded set may then be used to detect the type of exercise and the identity of the user.

The “type” of the exercise performed with the barbell 22 refers to the specific exercise, such as a squat, dead lift, bench press, overhead press, etc., performed with the barbell 22. However, embodiments are not limited to any specific types of exercises. The identity of the user that performed the exercise with the barbell 22 refers to a specific user identification, such as a name or identification number. For example, the identity of a user might be a name such as “John Smith”, “Sally Anderson”, or a number such as “24556”. However, embodiments are not limited to any specific manner of user identification.

Such embodiments can provide improved accuracy in detecting and counting repetitions as compared to conventional data analysis techniques and signal processing algorithms, by using both the identity of the user and the type of exercise as labels for a given set of data and by passing the labeled data set through at least one neural network. For example, such embodiments may leverage subtle differences between users in how an exercise is performed, to improve accuracy in detecting and counting repetitions. Moreover, such embodiments can be much less complex to design and implement, as compared to conventional data analysis techniques and signal processing algorithms.

Moreover, various embodiments can provide for the type of exercise and/or of the identity of the user to be quickly and easily detected from the data generated by the sensor 20. Various embodiments can also provide for sets and repetitions to be quickly easy detected from the data generated by the sensor 20. The detected information can be recorded and logged autonomously without requiring manual entry into a computer program and/or physical log book/sheet. Such embodiments provide significant technical benefits over conventional systems which either require a user or coach/trainer to manually enter the type of exercise and/or set information, or require strict adherence to predefined protocols.

In some embodiments, the type of the exercise performed with the barbell and the identity of the user that performed the exercise with the barbell might not be detected by a neural network, and instead might be provided as labeled data to the at least one neural network 24 to detect, or to detect and count, the repetitions using the labeled data.

Embodiments are not limited to detecting and counting repetitions. For example, the use of at least one neural network to detect the identity of a user that performed the exercise from data generated by a sensor coupled to a barbell is useful by itself. For example, as indicated above, such embodiments can provide for the identity of the user to be quickly and easily determined from motion data of the barbell, without requiring the identity of the user to be manually entered into a user interface or known based on sensor assignment in which a specific sensor is pre-assigned to a specific user or a specific group of users.

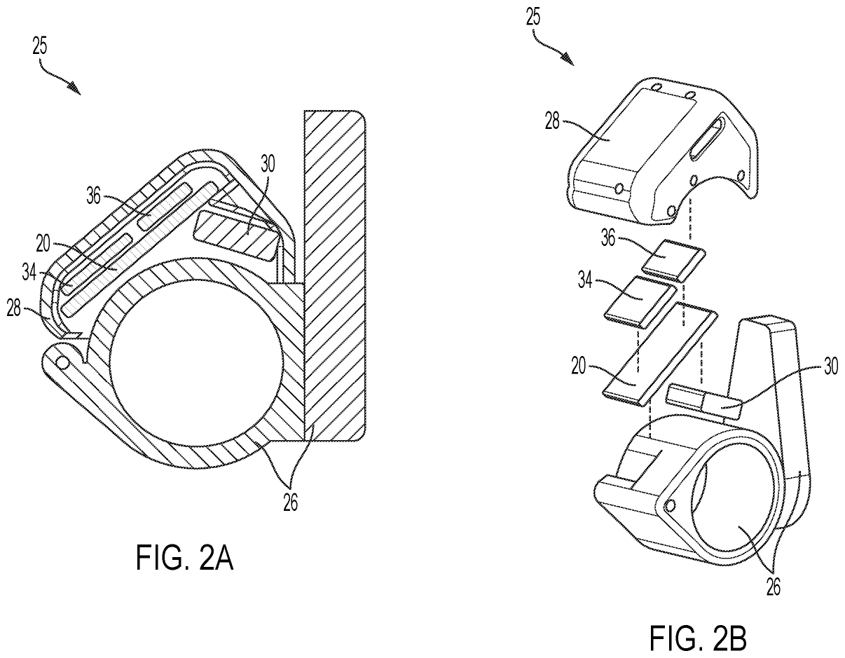

FIGS. 2A and 2B are diagrams illustrating a sensor housed in a housing, as a sensorized collar, according to an embodiment.

Referring now to FIGS. 2A and 2B, a sensorized collar 25 includes the sensor 20. The sensor 20 may include any combination of a single or multi-axis accelerometer, a single or multi-axis gyroscope, and a single or multi-axis magnetometer. In an embodiment, the sensor 20 included in the sensorized collar 25 is an inertial measurement unit (IMU) sensor that collects motion data.

The sensorized collar 25 may include a barbell collar 26 that may clamp to a barbell to thereby couple to the barbell. The sensorized collar 25 may also include a housing 28 that attaches to the barbell collar 26 or be integrally formed with the barbell collar 26. The housing 28 may house electronics, and provide protection and insulation to the electronics. Although this embodiment shows the use of a barbell collar to couple the sensorized collar 25 to a barbell, embodiments are not limited to any particular manner of coupling a senzorized collar, or a sensor, to a barbell.

In the embodiment in FIGS. 2A and 2B, the sensorized collar 25 may function as a collar to hold weights in place on the barbell. Moreover, instead a providing a sensorized collar 25, the sensor 20 may be coupled to the barbell 20 without providing any type of collar function. Accordingly, embodiments are not limited to the sensor functioning to hold weights in place on the barbell.

The sensorized collar 25 may further include a battery 30 that provides power, a microcontroller 34 that reads data from the sensor 20 and may transmit the data to a user's device and/or a server, and a voltage regulator 36 that may provide power regulation to ensure a safe interface between the battery 30 and the microcontroller 34.

However, the sensorized collar 25 is not limited to these specific components and/or to a specific configuration of these components, and many variations are possible. Moreover, the sensorized collar 25 is not limited to the specific shape shown in the figures, and many variations are possible.

The sensorized collar 25 may also include an interface to allow for manual inputs including, but not limited to, working load and/or exercise difficulty. In some embodiments, the sensorized collar 25 may include a touch screen and/or a dial indictor.

The sensorized collar 25 may include a feedback mechanism which would allow for audible, visible, or tangible feedback to be communicated to the user during an activity. Embodiments of feedback mechanisms may include, by are not limited to, a flashing light, a beep, and/or a vibration generator to communicate, for example, the completion of an event.

The sensorized collar 25 may include additional sensors including, but not limited to, a light detecting and ranging (LIDAR) sensor, a barometric pressure sensor, and/or radio frequency identification (RFID) sensor. Supplemental sensors, which are not coupled to the barbell, may be used in conjunction with the sensorized collar 25 to capture additional information. Such supplemental sensors may include, but are not limited to, load cells, pressure transducers, and/or camera/vision systems.

The sensor 20 may be coupled directly to the barbell to enable insights into unique motion profiles and performance data, which may include, but is not limited to, one or more of the following: (a) linear and angular acceleration and displacement, (b) orientation, (c) stability, (d) force output, (e) power output, and (f) asymmetries. Such data may be obtained using devices other than the sensor 20.

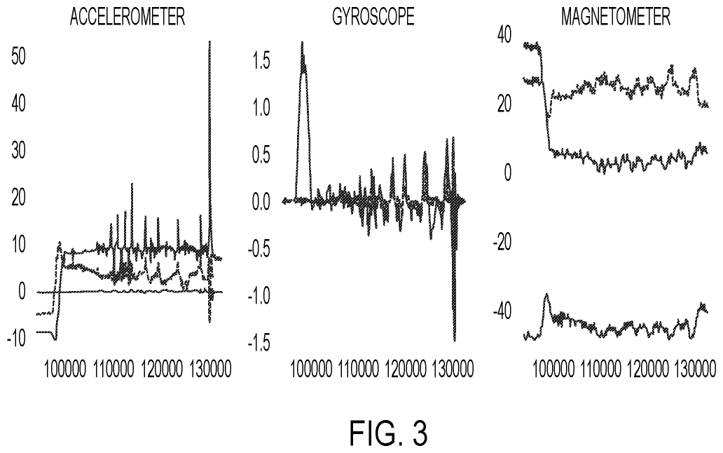

FIG. 3 is a diagram illustrating sample data from a prototype sensor implemented as a 9-axis IMU sensor, according to an embodiment. More specifically, the sample data in FIG. 3 was obtained for eight (8) bench press repetitions recorded using a prototype sensor coupled to a barbell and that included a nine (9) axis IMU without the housing of the sensor. As can be seen from FIG. 3, the sensor included an accelerometer, a gyroscope, and a magnetometer.

Various embodiments of a sensor and at least one neural network as described herein, may derive, for example, any combination of the following: (a) recognition of a working set being completed, (b) recognition of an individual repetition being completed, (c) identification of a specific user performing the working set, (d) identification of a unique barbell exercise being completed, including, but not limited to, squats, deadlifts, presses, rows, cleans, and variations thereof, (e) technique and form attributes and profiles including, by not limited to, acceleration, velocity, depth, tempo, stability, and asymmetry, and (f) force and power profiles of repetitions and/or sets.

Techniques for processing data generated by the sensor may include the use of, for example, mathematical algorithms and neural networks including, but not limited to, Recurrent Neural Networks (RNNs), fully connected neural networks, and Convolutional Neural Networks (CNNs). There are many different types and configurations of neural networks, and embodiments are not limited to any particular type of neural network and/or any particular configuration of a neural network. For example, CNNs are described herein in various embodiments, but an RNN or another type of neural network may by useable instead of a CNN. Similarly, RNNs are described herein in various embodiments, but a CNN or another type of neural network may by useable instead of a RNN. Moreover, neural networks can be configured to operate in series or in parallel, and embodiments are not limited to any specific configuration or number of neural networks.

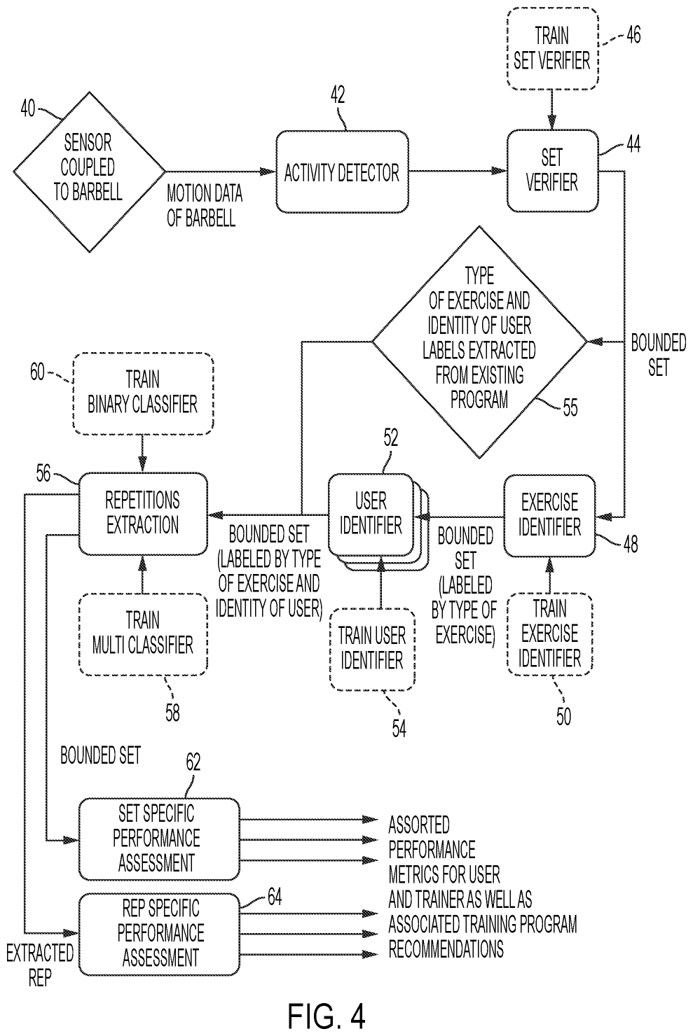

FIG. 4 is a diagram illustrating an example architecture of a system implementing a sensor and neural networks, according to an embodiment.

Referring now to FIG. 4, in operation 40, the sensor 20 is coupled to the barbell 22.

The sensor 20 may be, for example, a nine (9) axis IMU that generates motion data representable by, for example, a [9×n] matrix that includes horizontally stacked vectors that are in the form:

$$\vec{d} = [ \begin{matrix}{a}_{x} \\ {a}_{y} \\ {a}_{z} \\ {v}_{x} \\ {v}_{y} \\ {v}_{z} \\ {m}_{x} \\ {m}_{y} \\ {m}_{z}\end{matrix} ]$$

where aₖ is acceleration data, vₖ is angular velocity data, and mₖ is magnet field data.

However, the embodiments are not limited to a sensor having any specific number of axes or generating any specific data, and are not limited to data being represented by any specific vector, any specific matrix, etc.

The generated motion data may or may not capture data specific to a working set. Here, a “working set” is a set of motion data which captures all repetitions which occurred during a single activity performed with the barbell by a user without changing the type of exercise or taking a rest in which the barbell become static. In addition to capturing data specific to a working set, the generated motion data may also capture axillary activities, such as changing out weights, removing or placing the sensor on the barbell, or moving the sensor or barbell between different exercises. Moreover, the generated motion data may capture a static condition in which the barbell is not being moved.

Additional rows of inputs may be added to the motion data generated by the sensor 20, including filtered and/or processed data streams and labels For example, labels for a user performing the exercise and the exercise type may be added as additional inputs for each data stream.

In operation 42, an activity detector may be implemented by, for example, one or more processors that recognize barbell motion (activity) using, for example, a cumulative sum of change algorithm, which uses a threshold to determine whether changes in sensor data are significant. Data streams determined to be significant may be stored for further processing.

An example of a cumulative sum of change algorithm which may be used by an activity detector in operation 42 to determine significant activity is:

$$FOR EACH DATA POINT ( INDEX , i ) , \\ WHILE : {\sum }_{j = i}^{j = i + 50} ‖ {\vec{d}}_{j + 1} - {\vec{d}}_{j} ‖ \geq {k}_{threshold}$$

$$ACTIVITY IS OCCURING$$

However, embodiments are not limited to this specific cumulative sum of change algorithm, and many various are possible. Moreover, embodiments are not limited to the use of a cumulative sum of change algorithm to recognize barbell motion (activity), and other algorithms can be used.

Generally, the activity detector in operation 42 operates as a filter to limit the amount of data that is provided to subsequent processing. In other words, the activity detector limits the data to a range in which activity is detected, and provides this limited data downstream for subsequent processing.

In some embodiments, it may not be necessary to include an activity detector. In other words, operation 42 may not be provided, and all the data generated by the sensor 20 may be provided for downstream processing. However, the use of the activity detector in operation 42 can improve performance, accuracy, and speed of the processing of the architecture shown in FIG. 4, by limiting the amount of data needed to be processed by downstream neural networks.

In operation 44, a set verifier may be implemented, for example, by a RNN that classifies padded and masked variable length time-series of motion data labeled as either a 1 (working set) or a 0 (non-set), using a sigmoid activation function. However, embodiments are not limited to any particular type of neural network, or any particular type of activation function, or any particular classification technique, to verify a set. For example, an CNN may be used instead of a RNN in operation 44. Data sets which are classified by the set verifier as a “working sets” are herein referred to as “bounded sets”.

As indicated in operation 46, the set verifier, for example, a RNN, may be trained to verify sets using training data streams representing working sets and non-sets (i.e., weight changes or other extraneous barbell movement), labeled as either a 1 (working set) or a zero (non-set). However, embodiments are not limited to any particular manner of training a neural network as a set verifier.

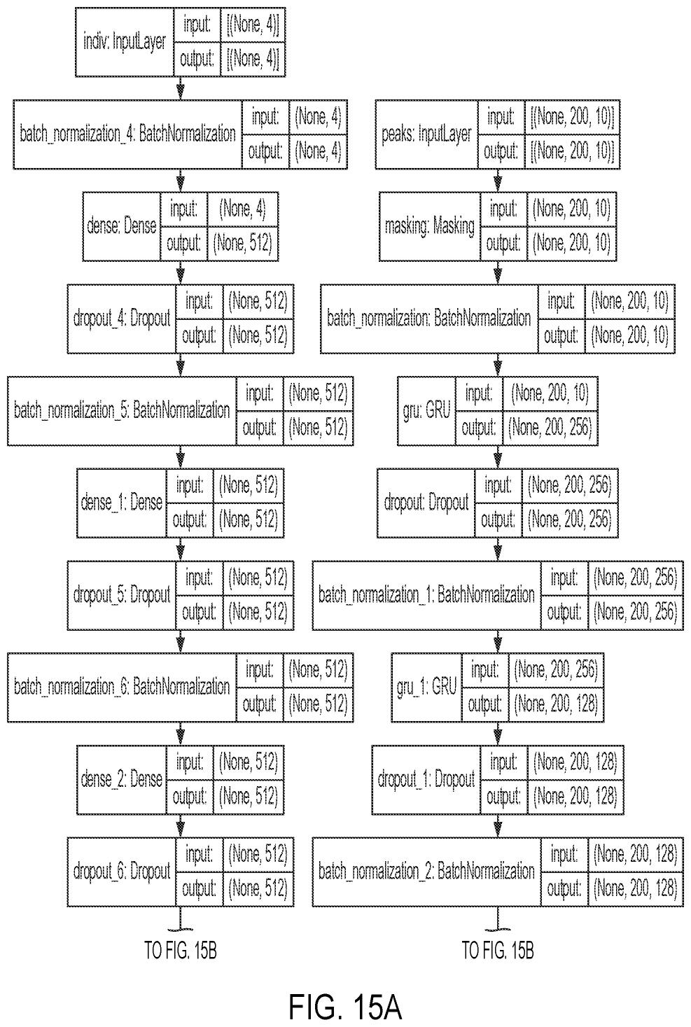

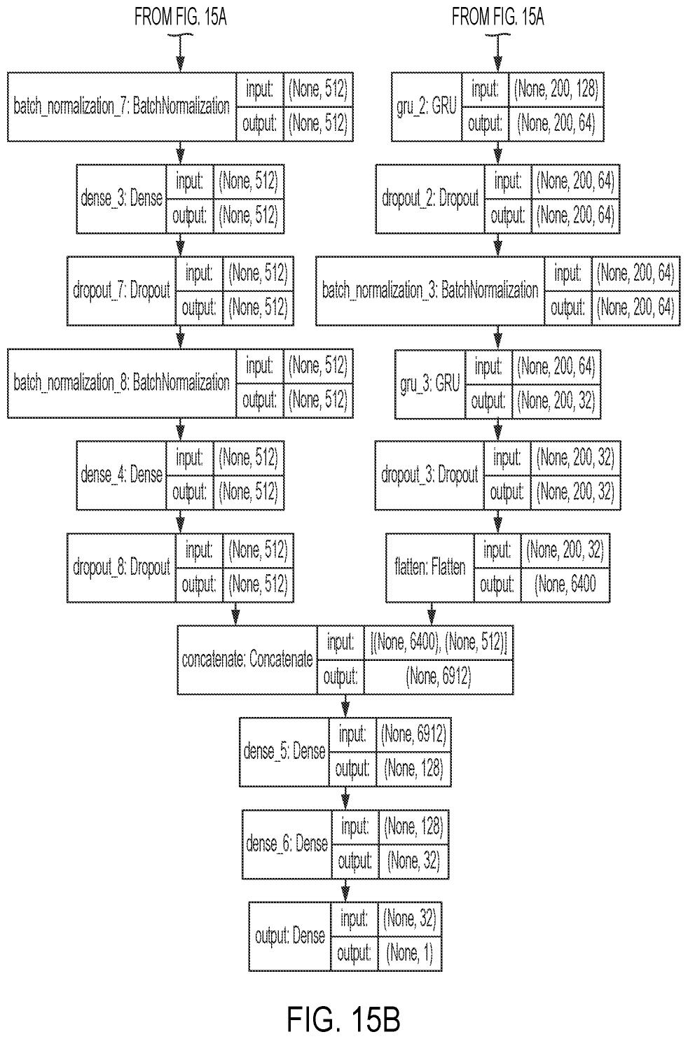

As indicated above, at least one neural network may be configured to classify motion data as a working set or non-set. The following is an example computer program that may be used to train a RNN KERAS model to detect working sets and label these as a 1 (true). In this example, local maxima (referred herein as peaks) are extracted from motion data using traditional data processing techniques and ten unique peak parameters are passed along with each peak datapoint as inputs. FIGS. 15A and 15B illustrate a plot of this example multiple input single output RNN model.

```
def train_model(peak_features, indiv_features, train_labels):
  dropout_rate = 0.1
  peaks_per =len (peak_features[0]) # 120 possible peaks per set
  n_input = len(peak_features[0][0]) # 10 input parameters per peak
  peaks_input = keras.Input(shape=(peaks_per,n _input), name=“peaks”)
  indiv_input = keras.Input(shape=(indiv_features.shape[1],), name=“indiv”)
  peak_layers = Masking(mask_value=0.0)(peaks_input)
  peak_layers = BatchNormalization( )(peak_layers)
  peak_layers = GRU(units=256, return_sequences=True)(peak_layers)
  peak_layers = Dropout(rate=dropout_rate)(peak_layers)
  peak_layers = BatchNormalization( )(peak_layers)
  peak_layers = GRU(units=128, return_sequences=True)(peak_layers)
  peak_layers = Dropout(rate=dropout_rate)(peak_layers)
  peak_layers = BatchNormalization( )(peak_layers)
  peak_layers = GRU(units=64, return_sequences=True)(peak_layers)
  peak_layers = Dropout(rate=dropout_rate)(peak_layers)
  peak_layers = BatchNormalization( )(peak_layers)
  peak_layers = GRU(units=32, return_sequences=True)(peak_layers)
  peak_layers = Dropout(rate=dropout_rate)(peak_layers)
  peak_layers = keras.layers.Flatten( )(peak_layers)
  indiv_layers = BatchNormalization( )(indiv_input)
  indiv_layers = Dense(units=512, activation=‘relu’)(indiv_layers)
  indiv_layers = Dropout(rate=dropout_rate)(indiv_layers)
  indiv_layers = BatchNormalization( )(indiv_layers)
  indiv_layers = Dense(units=512, activation=‘relu’)(indiv_layers)
  indiv_layers = Dropout(rate=dropout_rate)(indiv_layers)
  indiv_layers = BatchNormalization( )(indiv_layers)
  indiv_layers = Dense(units=512, activation=‘relu’)(indiv_layers)
  indiv_layers = Dropout(rate=dropout_rate)(indiv_layers)
  indiv_layers = BatchNormalization( )(indiv_layers)
  indiv_layers = Dense(units=512, activation=‘relu’)(indiv_layers)
  indiv_layers = Dropout(rate=dropout_rate)(indiv_layers)
  indiv_layers = BatchNormalization( )(indiv_layers)
  indiv_layers = Dense(units=512, activation=‘relu’)(indiv_layers)
  indiv_layers = Dropout(rate=dropout_rate)(indiv_layers)
  x = keras.layers.concatenate([peak_layers, indiv_layers])
  x = Dense(units=128 activation=‘relu’)(x)
  x = Dense(units=32, activation=‘relu’)(x)
  x_pred = Dense(1, activation=‘sigmold’, name=“output”)(x)
  model = keras.Model(
    inputs=[peaks_input; indiv_input] ,
    outputs=[x_pred] ,
  )
  model.compile(
    optimizer=keras.optimizes.Adam(learning_rate=0.001, beta_1=0.9, beta_2=0.999),
    metrics=[“accuracy”],
    loss = keras.losses.BineryCrossentropy(from_logits=True),
  )
  model.fit(
    {“peaks”: peak_features, “indiv”: indiv_features},
    {“output”: train_labels},
    epochs = num_epochs,
    batch_size=64,
  )
```

Of course, embodiments are not limited to the above example computer program or the plot shown in FIGS. 15A and 15B, and are not limited to any number of unique peak parameters or to a multiple input single output model. Moreover, the above computer program uses KERAS, which is a deep learning application programming interface (API) written in PYTHON, running on top of the machine learning platform TENSORFLOW. However, embodiments are not limited to computer programs using KERAS, PYTHON, or TENSORFLOW, and other APIs, languages and platforms can be used. Moreover, the above computer program implements an RNN, but other types of neural networks can be implemented. Further, the structure of the above computer program is only one example, any many variations are possible.

In operation 48, an exercise identifier detects a type of exercise performed with the barbell from the data generated by the sensor 20. An exercise identifier may be implemented, for example, by a RNN operating as a multi-classifier to classify padded and masked variable length time-series of motion data using, for example, a softmax activation function, to classify a bounded set as one of multiple potential exercise types. The RNN may add a label to motion data of a bounded set to indicate the type of exercise to which the bounded set was classified. However, embodiments are not limited to any particular type of neural network, or any particular type of activation function, or any particular classification technique, to classify a bounded set to one of multiple potential exercise types. For example, a CNN may be used instead of a RNN in operation 48.

As indicated in operation 50, the exercise identifier, for example, a RNN, may be been trained to classify a bounded set into one of multiple potential types of exercises using training data representing bounded sets, labeled as one of multiple possible types of exercises. However, embodiments are not limited to any particular manner of training a neural network to classify a bounded set as a type of exercise.

In operation 52, a user identifier detects an identity of a user that performed the exercise with the barbell from the data generated by the sensor 20 and, in some embodiments, with the type of exercise indicated by the label provided by operation 48. A user identifier may be implemented, for example, by a CNN operating as a multi-classifier to classify a bounded set, labeled by type of exercise, as one of multiple potential users using a softmax activation function. The CNN may add a label to motion data of a bounded set to indicate the identity of the user to which the bounded set was classified. However, embodiments are not limited to any particular type of neural network, or any particular type of activation function, or any particular classification technique, to classify a bounded set to one of multiple potential users.

As indicated in operation 54, the user identifier, for example, a CNN, may be trained to classify a bounded set into one of multiple potential users using training data representing bounded sets, labeled as one of multiple possible users and, in some embodiments, as one of multiple types of exercises. However, embodiments are not limited to any particular manner of training a neural network to classify a bounded set as a type of exercise.

Although a neural network is used in operation 48 to identify a type of exercise and in operation 52 to identify a user, a type of exercise and an identity of a user may instead be extracted from an existing program in operation 55. In other words, the type of exercise and the identity of a user may be provided via labeled data, instead of having that the type of exercise and the identity of the user be extracted from the data generated by the sensor 20.

For example, in operation 55, the type of exercise and the identity of a user may be extracted from labeled data provided by an existing computer program. The labeled data, provided by the existing program, may also be used, in some embodiments, to cross check the predictions made by the user identifier in operation 52 and the exercise identifier in operation 48.

Moreover, operations 48 and 52 may be reversed, so that a first neural network detects the identity of the user and provides a label indicating the identity of the user, and then a second neural network determines the type of exercise using the identity of the user indicated by the label. In such an embodiment, the first neural network would be trained with training data labeled with the identity of the user, the second neural network would be trained with training data labeled with the identity of the user and the type of exercise.

Further, in some embodiments, the identity of the user may be extracted in other manners, including but not limited to, an RFID or other form of tag, or via a dedicated sensor already linked to a specific user. In such embodiments, the user identifier in operation 52 may be replaced with a label corresponding to the identity of the user. In some embodiments, the label for the identity of the user may be passed directly into the exercise identifier in operation 48 along with the corresponding motion data. The exercise identifier in operation 48 in such embodiments would be trained using motion data labeled with both the type of exercise and the identity of the user.

Moreover, in some embodiments, the user identifier in operation 52 may include multiple neural networks, rather than, for example, a single neural network. For example, in some embodiments, the user identifier in operation 52 may include a unique user identifier neural network for each unique exercise. In such an embodiment, a separate neural network may be used to classify users for one type of exercise (e.g., back squats) compared to a neural network to classify users for a different type of exercise (e.g., bench presses). Similarly, in an embodiment where operations 48 and 52 are reversed as described above, the exercise identifier may include a unique exercise identifier for each unique user. Then, once the user is predicted by the user identifier, a unique neural network may be used to determine which exercise was performed by that specific user.

In operation 56, a repetition extractor detects and counts the repetitions which occurred within a bounded set.

For example, in some embodiments, in operation 56, the repetition extractor may include at least one neural network that detects the repetitions which occur within a bounded set, and the detected repetitions can then be counted.

In other embodiments, in operation 56, the repetition extractor may include at least one neural network that predicts the total number of repetitions which occur within a bounded set of an exercise performed by a user with a barbell. For example, the repetition extractor may include at least one trained neural network that is trained using labeled data indicating the total number of repetitions which are contained within a bounded set. In such an embodiment, the at least one neural network of the repetition extractor may determine the number of repetitions within a bounded set. In other words, in such embodiments, the repetition extractor may detect and count the number of repetitions, without first detecting the repetitions and subsequently counting the detected repetition.

A parallel repetition detection technique may be used in operation 56 to improve accuracy in detecting completed repetitions within a bounded set. For example, the repetitions extractor in operation 56 may be implemented by, for example, (a) an RNN operating as a binary classifier using, for example, sigmoid activation function that is trained to determine if data corresponding to a given timestep represents the completion of an executed repetition for a specific type of exercise, and thereby detect repetitions, (b) a CNN operating as a multi-classifier using, for example, a softmax activation function, that is trained to detect the number of repetitions executed within a bounded set of data, and (c) processing to compare repetitions detected using the RNN and repetitions detected using the CNN against one another and against a prescribed program, if available, and flagging disagreements for further investigation, to confirm the detected repetitions and count the repetitions.

Therefore, in operation 56, data of a bounded set and labels, including the type of exercise and the identity of a user, may be passed through both the CNN and the RNN to detect and count repetitions in a parallel repetition detection technique.

In operation 58, the multi-classifier, for example, a CNN, may be trained using training data of bounded sets, labeled with the total number of repetitions completed as part of the set.

In operation 60, the binary classifier, for example, a RNN, may be trained using training data of bounded sets, specific to a given type of exercise, labeled with one-hot vectors applied to timesteps immediately after the completion of a repetition and zeros elsewhere.

It should be understood that operations 56, 58 and 60 are not limited to any particular type of neural network, or any particular type of activation function, or any particular classification technique, to detect and count repetitions.

Moreover, embodiments are not limited to any particular manner of implementing parallel repetition detection techniques, and are not limited to the use of any particular types of neural networks for such parallel repetition detection techniques. Moreover, embodiments are not limited to parallel repetition detection. For example, a single RNN or CNN, or another type of neural network, may be implemented to detect repetitions in operation 56.

In operation 62, various set specific performance assessments may be determined.

In operation 64, various repetition specific performance assessments may be determined.

For example, in operations 62 and 64, various assorted performance metrics may be determined, and used to provide performance assessments. Mathematical models, computations, filtering techniques, and/or artificial intelligence (AI) networks may be applied to the derived performance metrics.

As indicated above, at least one neural network may be configured to detect and count repetitions. The following is an example computer program that may be used to implement a RNN KERAS model to detect and count repetitions in operation 56:

X_input = Input(shape = input_shape)
# ID CONV layer
X = Conv1D(filters=44,kernel_size=4,strides=4)(X_input) # CONV1D

| | |
|---|---|
| X = BatchNormalization( )(X) | # Batch normalization |
| X = Activation(“relu”)(X) | # ReLu activation |
| X = Dropout(rate=0.2)(X) | # dropout |
| # First GRU Layer | |
| X = (GRU(units=44, return_sequences = True))(X) | # GRU (44 units) |
| X = Dropout(rate = 0.2) (X) | # dropout |
| X = BatchNormalization( )(X) | # Batch normalization |
| # Second GRU Layer | |
| X = (GRU(units=28, return_sequences = True))(X) | # GRU (28 units) |
| X = Dropout(rate = 0.2)(X) | # dropout |
| X = BatchNormalization( )(X) | # Batch normalization |
| X = Dropout(rate = 0.2)(X) | # dropout |
| # Time-distributed dense layer | |

```
X = TimeDistributed(Dense(1, activation = “sigmoid”))(X) # time distributed
model = Model(inputs = X_input, outputs = X)
return model
# Training the Model
model = model(input_shape = (n_steps, n_input))
loss=[ ] # list to store loss over multiple iterations of training
accuracy=[ ] # list to store accuracy over multiple iterations of training
f1=0[ ] # list to store f1 over multiple iterations of training
for i in range(3):
  opt = Adam(Ir=0.01*10**-int(i/3), beta_1=0.9, beta_2=0.999, decay=0.01)
  model.compile(loss=‘binary_crossentropy’, optimizer=opt, metrics=[“accuracy”, get_f1]
)
history = (model.fit(x_train, y_train, batch_size = 23, epochs=400))
# Testing the model on Dev Set
loss, acc, f1 = model.evaluate(x_dev, y_dev)
```

Of course, the above computer program is only one example, and many variations are possible. For example, the above computer program uses KERAS, which is a deep learning application programming interface (API) written in PYTHON, running on top of the machine learning platform TENSORFLOW. However, embodiments are not limited to computer programs using KERAS, PYTHON, or TENSORFLOW, and other APIs, languages and platforms can be used. Moreover, the above computer program implements an RNN, but other types of neural networks can be implemented. Further, the structure of the above computer program is only one example, any many variations are possible.

The architecture in FIG. 4 is only one example architecture, and many variations are possible.

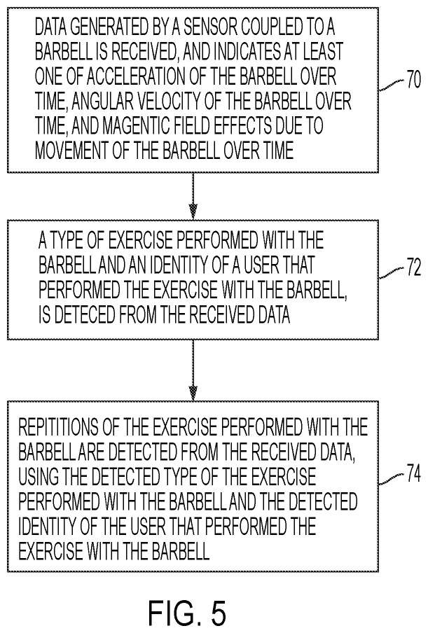

For example, FIG. 5 is a diagram illustrating a process of detecting repetitions of an exercise performed with a barbell, from data generated by a sensor coupled to the barbell, according to an embodiment. The process in FIG. 5 implements various, but not all, of the operations in FIG. 4.

Referring now to FIG. 5, in operation 70 data generated by the sensor 20 coupled to barbell 22 is received. The received data indicates at least one of acceleration of the barbell over time, angular velocity of the barbell over time, and magnetic field effects due to movement of the barbell over time.

In operation 72, a type of exercise performed with the barbell and an identity of a user that performed the exercise with the barbell is detected from the received data.

In operation 74, repetitions of the exercise performed with the barbell are detected from the received data, using the detected type of exercise performed with the barbell and the detected identity of the user that performed the exercise with the barbell.

Therefore, the embodiment in FIG. 5 may be implemented using, for example, operations 48, 52 and 56 in FIG. 4, although the embodiment in FIG. 5 is not limited to using these operations from FIG. 4, and many variations are possible.

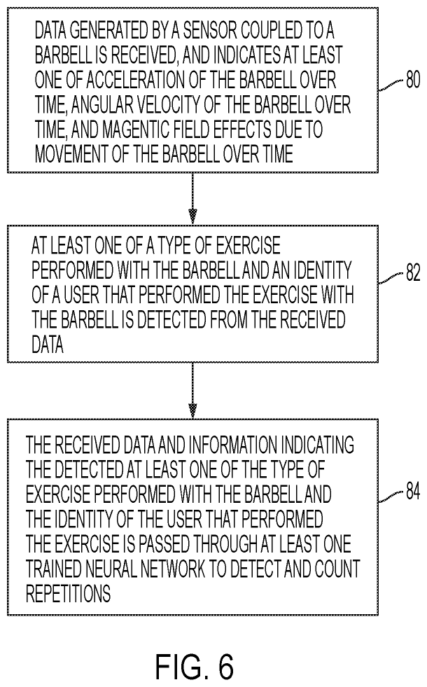

FIG. 6 is a diagram illustrating a process of detecting and counting repetitions of an exercise performed with a barbell, according to an additional embodiment.

Referring now to FIG. 6, in operation 80, data generated by the sensor 20 coupled to the barbell 22 is received. The received data indicates at least one of acceleration of the barbell over time, angular velocity of the barbell over time, and magnetic field effects due to movement of the barbell over time.

In operation 82, at least one of a type of exercise performed with the barbell and an identity of a user that performed the exercise with the barbell is detected, from the received data. For example, in embodiment, the type of exercise performed with the barbell may be detected, but the identity of the user that performed the exercise with the barbell may not be detected. In another embodiment, the identity of the user that performed the exercise with the barbell may be detected, but the type of exercise performed with the barbell may not be detected. In another embodiment, both the type of exercise performed with the barbell and the identity of a user that performed the exercise with the barbell is detected. In various embodiments, the type of exercise performed with the barbell and/or the identity of the user that performed the exercise with the barbell may be passed as inputs, rather than being detected from data generated by the sensor.

In operation 84, the received data and information indicating the detected at least one of the type of exercise performed with the barbell and the identity of the user that performed the exercise is passed through at least one trained neural network, to detect and count repetitions of the exercise performed with the barbell by the user.

As an example, if type of exercise performed with the barbell is detected in operation 82, but the identity of the user is not detected in operation 82, then the received data information and information indicating the type of exercise performed with the barbell is passed through at least one trained neural network in operation 84 to detect and count repetitions from the type of exercise performed with the barbell. Such an embodiment would implement, for example, operations similar to operations 48 and 56 in FIG. 4, but would not implement operation 52 in FIG. 4. With such a configuration, the repetition extractor in operation 56 would be trained to detect and count repetitions using detected type of exercise, but not the identity of the user. Such training would be performed by training a neural network with training data labeled with the type of exercise.

Similarly, as an example, if the identity of the user is detected in operation 82, but the type of exercise performed with the barbell is not detected in operation 82, then the received data and information indicating the identity of the user is passed through at least one trained neural network in operation 84 to detect and count repetitions from the identity of the user. Such an embodiment would implement, for example, operations similar to operations 52 and 56 in FIG. 4, but would not implement operation 48 in FIG. 4. With such a configuration, the repetition extractor in operation 56 would be trained to detect and count repetitions using detected identity of the user, but not the type of exercise. Such training would be performed by training a neural network with training data labeled with the identity of the user.

As an additional example, if both the identity of the user and the type of exercise performed with the barbell are detected in operation 82, then the received data and information indicating the identity of the user and information indicating the type of exercise performed with the barbell is passed through at least one trained neural network in operation 84 to detect and count repetitions from the identity of the user and the type of exercise. Such an embodiment would implement, for example, operations 48, 52 and 56 in FIG. 4.

Therefore, the embodiments in FIG. 6 may be implemented using operations similar to various operations in FIG. 4, although the embodiment in FIG. 6 is not limited to using specific operations from FIG. 4, and many variations are possible.

As indicated above, in operation 84, repetitions are detected and counted. For example, in an embodiment, in operation 84, the received data and information indicating the detected at least one of the type of exercise performed with the barbell and the identity of the user that performed the exercise is passed through at least one trained neural network, to (a) determine, for each repetition of the exercise performed with the barbell by the user, when the repetition occurs, to thereby detect each repetition of the plurality of repetitions, and (b) count the detected repetitions.

As an additional example, in another embodiment, in operation 84, at least one neural network can be implemented to detect and count the repetitions, instead of first detecting the repetitions and then subsequently counting the detected repetitions.

Referring again to FIG. 4, as indicated in operations 62 and 64 in FIG. 4, various set specific performance assessments and repetition specific performance assessments can be determined.

One such performance assessment may be an intensity index. More specifically, a rate of exertion may be predicted by learning and recognizing tendencies related to exercise difficulty and/or fatigue including, but not limited to velocity and stability.

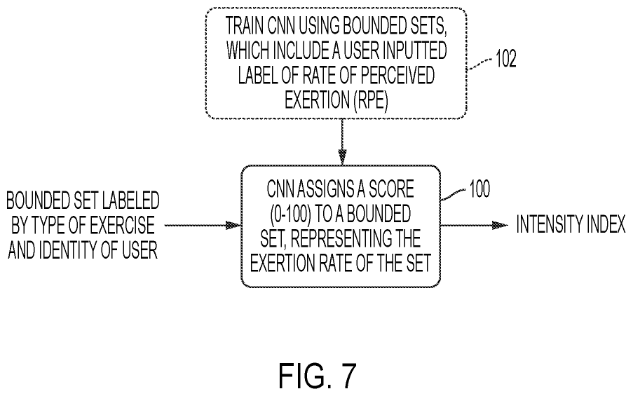

FIG. 7 is a diagram illustrating the determination of performance metric referred to herein as an intensity index and that can be used for performance assessment, according to an embodiment.

Referring now to FIG. 7, in operation 100, an intensity index calculator may use, for example, a CNN to assign a score between, for example, 0 and 100, to a bounded set which is labeled by type of exercise and identity of a user as inputs, although the neural network is not limited to any particular type of neural network. The assigned score represents an exertion rate of the set. As indicated in operation 102, the CNN may be trained, for example, on training data of bounded sets labeled by a user or trainer with a Rate of Perceived Exertion between 0 and 100.

Of course, the determination of an intensity index is not limited to the specific embodiment in FIG. 7, and many variations are possible.

Another performance assessment that may be determined is a silk score, which refers to Smoothness in Lifting Kinematics (SILK).

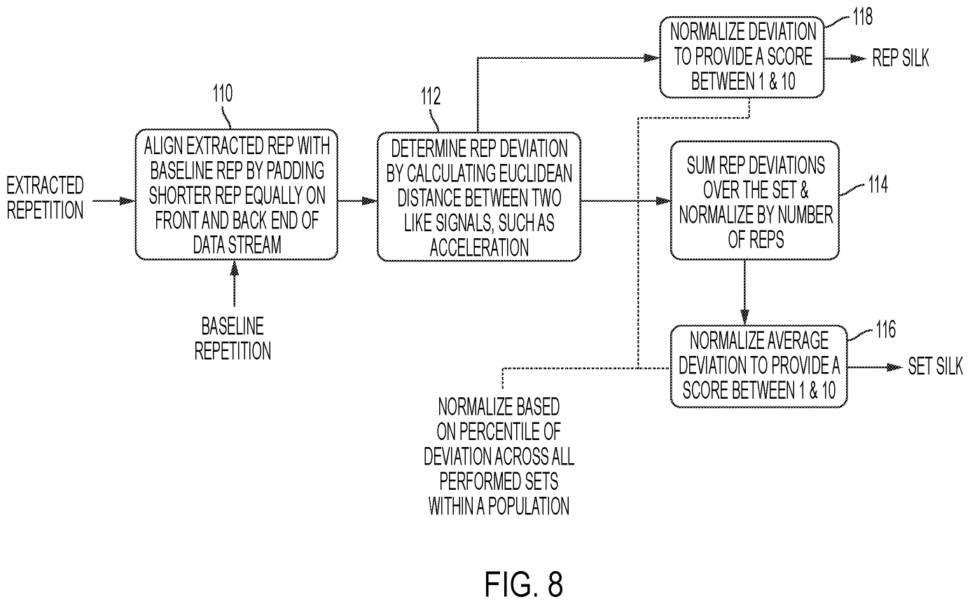

FIG. 8 is a diagram illustrating the determination of a performance metric which may be referred to herein as a silk score and that can be used for performance assessment, according to an embodiment. The silk score is a metric which may be used to communicate how precise and smooth a set appears, based on adherence in execution, by comparing the current working set or repetition against a baseline set or repetition which is of known high quality (associated to a lighter working load or evaluated via trainer or other methods). The silk score may consider, for example, tempo, velocity, and/or range of motion, and represents the deviation in set and/or repetition quality compared to a standard performance.

Referring now to FIG. 8, one embodiment of a silk score calculator uses, for example, a Euclidean distance algorithm to determine the deviation of a given repetition against a baseline repetition.

euclidean(a^{(b)},a^{(c)})={square root over (Σᵢ(aᵢ^{(b)}−aᵢ^{(c)})²)}

In operation 110, an extracted repetition specific to a bounded set, and a baseline repetition are provided as inputs. The baseline repetition may have been extracted from a previous set of the same exercise type, performed by the same user, with a lighter working load. The baseline repetition may be identified by a program, user, coach, or trainer as being a well-executed repetition. With these inputs, as indicated in operation 110, an extracted repetition is aligned with the baseline repetition by padding shorter repetitions equally on front and back ends of a data stream.

In operation 112, a repetition deviation may be determined by calculating Euclidean distance between two signals, such as acceleration.

In operation 114, the repetition deviations over the set are summed and normalized by the number of repetitions.

In operation 116, an average deviation can be normalized to provide a score between, for example, 1-10, but it not limited to these values. The normalization can be based, for example, on percentile of deviation across all performed sets with in a population. The normalized score can be used in some embodiments to determine a silk score for a set.

In operation 118, the determined repetition may be normalized to provide a score between, for example, 1 and 10, but it not limited to these values. The normalized score can be used in some embodiments to determine a silk score for a repetition.

Of course, the determination of a silk score is not limited to the specific embodiment in FIG. 8, and many variations are possible.

Another performance assessment that may be determined is a form factor, which is a metric used to capture and qualify the quality of exercise technique. The form factors may consider, for example, tempo, depth, asymmetry, stability, and/or velocity profiles to estimate the quality in exercise technique.

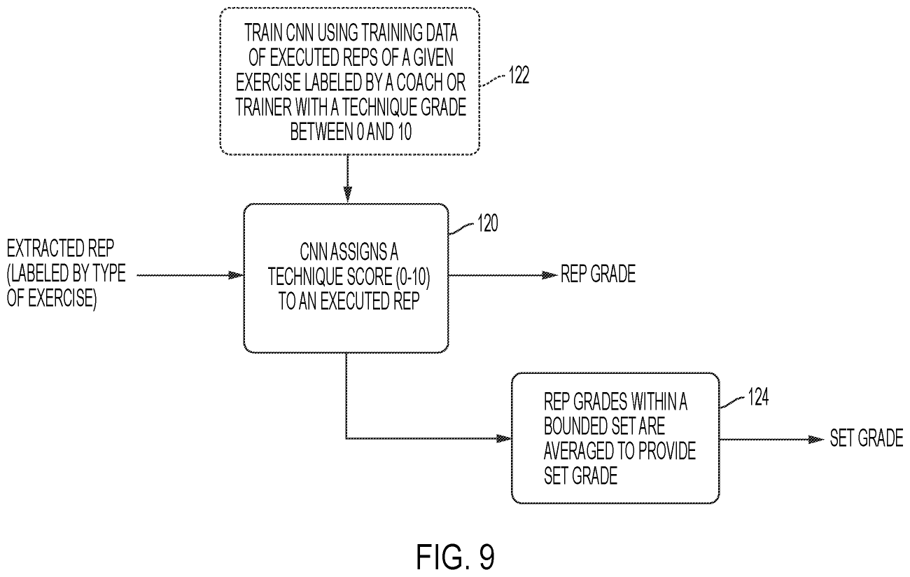

FIG. 9 is a diagram illustrating the determination of a performance metric referred to herein as a form factor and that can be used for performance assessment, according to an embodiment.

Referring now to FIG. 9, in operation 120, a form factor calculator uses, for example. a CNN to assign a score between, for example, 0 and 10, to an extracted repetition labeled by type of exercise as an input, although the neural network is not limited to any particular type of neural network.

In operation 122, the CNN may be trained using training data of executed repetitions of a given exercise labeled by, for example, a coach or trainer with a technique grade between, for example, 0 and 10.

In operation 124, repetition grades within a bounded set may be averaged to provide a set grade.

Of course, the determination of a form factor is not limited to the specific embodiment in FIG. 9, and many variations are possible.

Another performance assessment that may be determined is a force factor, which is a metric that may be used to capture the force generated by the user throughout the progression of an executed repetition, specifically concerning force generation specific to unique phases of the repetition (i.e., a first pull of a snatch or clean).

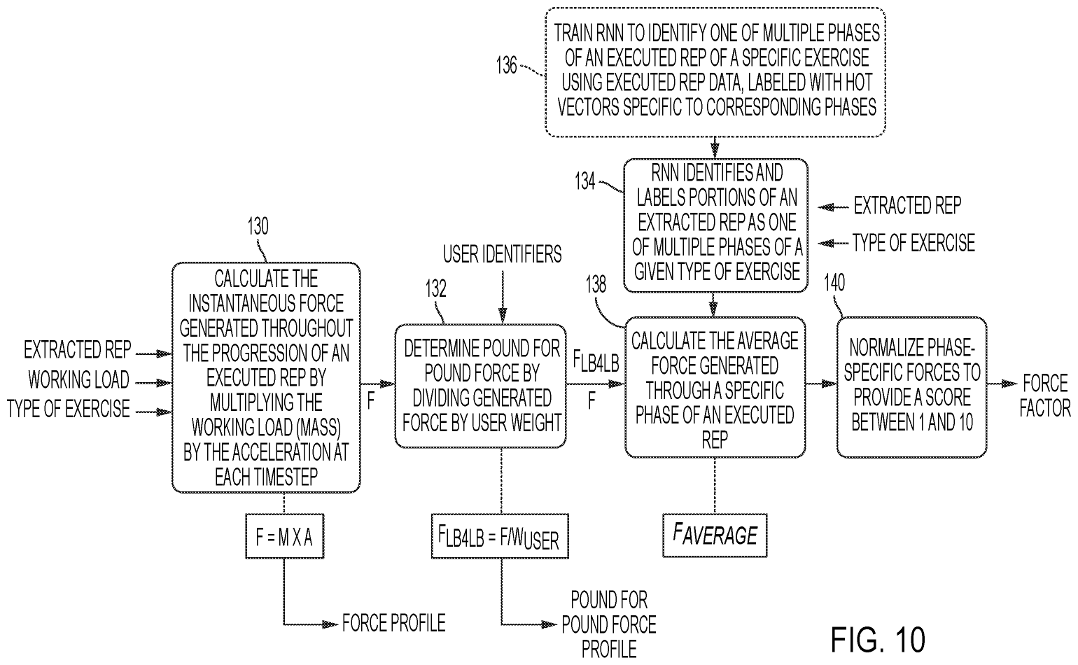

FIG. 10 is a diagram illustrating the determination of a performance metric referred to herein as a force factor and that can be used for performance assessment, according to an embodiment.

In an embodiment such as that in FIG. 10, a force factor calculator uses a neural network to recognize and isolate unique phases of a given executed repetition of a specific exercise and then calculates the average force generated within each phase, using the force profile (absolute force and/or pound-for-pound force) of the executed repetition.

Referring now to FIG. 10, in operation 130, the instantaneous force generated throughout the progression of an executed repetition is calculated by multiplying the working load (mass) by the acceleration at each timestep. As a result, a resulting force profile can be provided.

In operation 132, pound for pound force (F_{lb4lb}) is determined by dividing the force by weight of the user. As a result, a pound for pound force profile can be provided. The force factor can be determined by normalizing total force and/or pound for pound force with consideration of attributes such as weight, height, wingspan, hip weight, body mass index (BMI), body fat percentage, age, sport, position, team, divisional level.

In operation 134, a neural network, such as, for example, an RNN, identifies and labels portions of an extracted repetition as one of multiple phases of a given exercise.

As indicated in operation 136, the RNN may be trained to detect one of multiple phases of an executed repetition of a specific exercise using executed repetition data, labeled with hot vectors specific to corresponding phases (i.e., data relating to the “first pull” is labelled with 1s).

In operation 138, an average force generated through a specific phase of an executed repetition (i.e., an “eccentric phase” or a “first pull”) is calculated, where:

$$\vert {F}_{average} = {\sum }_{i} {F}_{i} + i .$$

In operation 140, phase-specific forces are normalized to provide a score between, for example, 1 and 10. Such normalization can be based, for example, on percentile of force generation metrics across all performed sets of a given exercise within a population. The population would be based, for example, on above described attributes such as weight, height, wingspan, hip weight, body mass index (BMI), body fat percentage, age, sport, position, team, divisional level.

Of course, the determination of a force factor is not limited to the specific embodiment in FIG. 10, and many variations are possible.

Another performance assessment that may be determined is a power profile, which is a metric that expands upon the force profiles and isolated repetition phases to calculate the rate of force development (RFD) within a specific phase of an executed repetition.

One embodiment of an RFD calculator within a given phase of an executed repetition divides the maximal instantaneous force generated within the phase by the time taken to reach the maximal force, as indicated by the following equation.

Fₘₐₓ/T_{Fmax}.

Another performance assessment that may be determined is a twitch rating, which is a normalized metric which considers force factor and RFD to provide insight into explosiveness of the user.

Synthesis of the above metrics allows for a comprehensive grade to provide holistic feedback on the exercise execution.

Through the utilization of data analytics applied to a pre-defined system, a community network can be used to facilitate collaboration and competition amongst users of various populations and groups. This platform would allow users to compare metrics and performance results against other users of specific groups to which one belongs, such as a user's athletic team, program, or league. Comparison feedback could be communicated as an absolute value or percentage rank. Performance metrics could also be normalized based on physical or athletic attributes for competition and comparison among people of different body types and goals. Normalized metrics provide insights into the user's relative proficiency relative to their peers or competitors.

Attributes used for normalization, herein referred to as user identifiers include, for example: (a) physical attributes, such as age, height, weight, wingspan, hip height, BMI, or body fat percentage, and (b) user attributes, such as sport, position, team, program, league, division, or level.

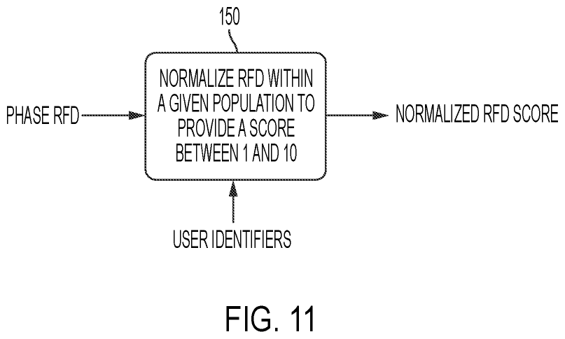

For example, FIG. 11 is a diagram illustrating a performance metric referred to herein as normalized RFD score and which can be used for performance assessment, according to an embodiment. The normalized RFD score is a normalized metric that considers the RFD of a user for the first pull of a power clean repetition as a percentile of all users who play the same sport and the same position at the same level (i.e., all D1 collegiate baseball third basemen), according to an embodiment. The percentile ranking corresponds to a normalized RFD score between, for example, 1 and 10.

Referring now to FIG. 11, in operation 150, a phase RFD (for example, the rate of force development for the first pull of an extracted power clean repetition at a given working load) is normalized within a given population to provide a score between, for example, 1 and 0, by determining the percentile which the RFD ranks within a given population (i.e., all D1 collegiate baseball third basemen) for the same type of exercise and working load.

Of course, the determination of a normalized RFD score is not limited to the specific embodiment in FIG. 11, and many variations are possible.

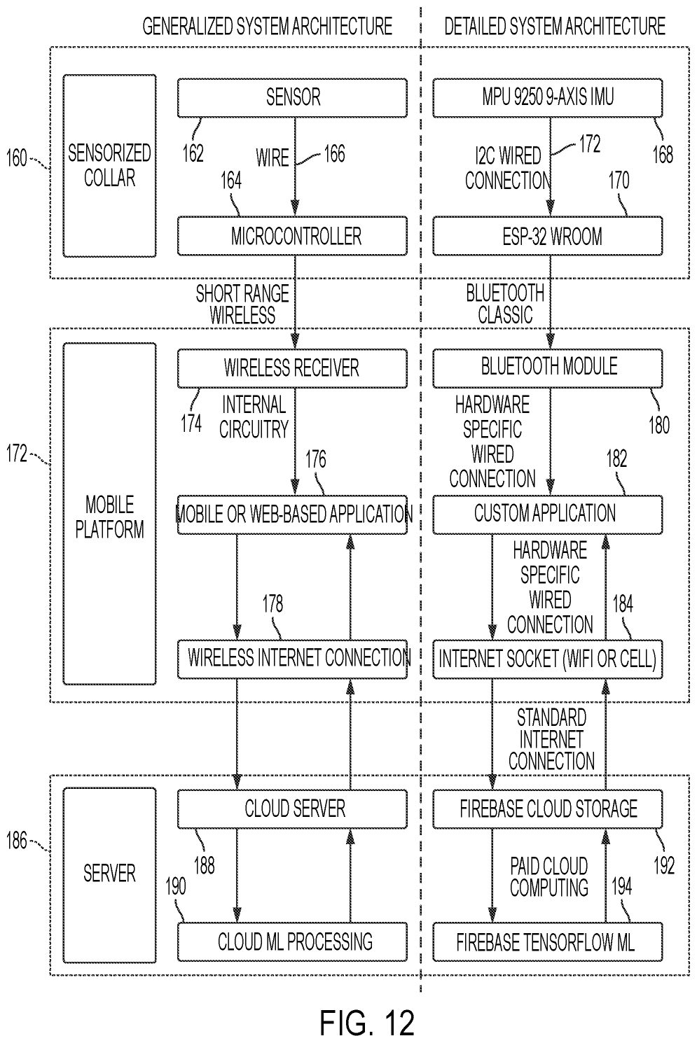

FIG. 12 is a diagram illustrating a generalized system architecture and a detailed system architecture, according to an embodiment.

Referring now to FIG. 12, in the generalized system architecture, a sensorized collar 160 may include a sensor 162 and a microcontroller 164 connected by a wire 166. In the detailed system architecture, the sensor might be, for example, a MPU 9250 9-axis IMU 168, the microcontroller might be, for example, an ESP-32 WROOM 170, and the wire might be, for example, in 120 wired connection 172.

In the generalized system architecture, a mobile platform may include a wireless receiver 174, a mobile or web-based application 176 and a wireless internet connection 178. In the detailed system architecture, the wireless receiver may be, for example, a BLUETOOTH module 180, the mobile or web-based application may be, for example, a custom application 182, and the wireless internet connection may be, for example, an internet socket (WiFi or cell).

The mobile platform 172 may be, for example, a mobile phone (including, for example, a smartphone), a laptop computer, a tablet computer, a wearable computing device and/or any other type of mobile device capable of performing computing operations to implement embodiments described herein. However, embodiments are not limited to the use of a “mobile” platform, and the platform can be, for example, a desktop computer or other computing device which would typically not be referred to as “mobile”. Therefore, a more general “computing device” can be used instead of the “mobile” platform 172. Such a computing device may include mobile devices, and other computing devices which are not typically referred to as “mobile”.

In the generalized system architecture, a server 186 may include a cloud server 188 and cloud machine learning (ML) processing 190. In the detailed system architecture, the cloud server might be, for example, a FIREBASE cloud storage 192, and the cloud ML processing might be, for example, FIREBASE TENSORFLOW ML processing 194.

The components shown in the detailed system architecture are commercially available products, and embodiments are not limited to any specific commercially available products.

Moreover, the generalized system architecture and the detailed system architecture in FIG. 12 show the sensorized collar 160 communicating with the mobile platform 172 through short range wireless communication, and the mobile platform 172 communicating with the server 186 over an internet connection. However, embodiments are not limited to any particular communication technology or type of connection, and the lines of communication are not limited to the sensorized collar 160 communicating with the mobile platform 172, and the mobile platform 172 acting as an in-between device to communicate with both the sensorized collar 160 and the server 172, as in FIG. 12. Instead, the sensorized collar 160, the mobile platform 172 and the server 186 may each be able to communicate with each other through various communication protocols and configurations.

Moreover, embodiments are not limited to the user of a mobile platform 172 and a server 186. Instead, processing can be distributed and/or configured in many different embodiments.

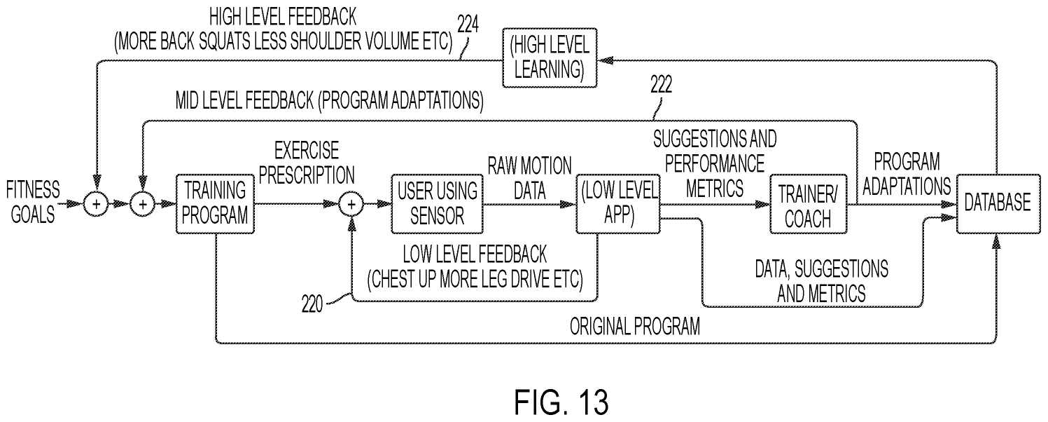

Moreover, the generalized system architecture and the detailed system architecture shown in FIG. 13 are only example embodiments, and embodiments are not limited to any specific configuration and/or any specific components. Instead, many variations are possible. Moreover, the generalized system architecture and the detailed specific architecture show various connections as being wired or wireless, but embodiments are not limited to any particular type of connection.

A system can be implemented to provide optimization of a user's program and to provide coaching assistance. More specifically, through the aggregation of data collected across a population of users, the system may map characteristics of training routines to performance metrics. This mapping enables recognition of deficiencies or weaknesses in an individual user's performance, approaching plateaus, and injury risk. Once recognized, deficiencies and risks may be corrected through recommendations of training modifications that maximize training efficiency and lead to performance gains.

Examples of recommendations derived by the system may include, but are not limited to, exercise selection and variations, work volume, weight ranges, technique alterations, and loading progression.

FIG. 13 is a diagram illustrating a system of data aggregation, mapping, and recommendation, according to an embodiment. The system in FIG. 13 includes a series of three control feedback loops which include the user, coach, and the automated data analysis system. In controls terminology, one can consider the user and their workout the plant, on which data is collected and feedback is provided via a training plan, which takes the role of the controller, although embodiments are not limited to any specific number of control feedback loops and/or any specific personnel included in the feedback loops.

Referring now to FIG. 13, in a first feedback loop 220, data is collected locally, and low-level feedback is provided to the user via the application interface. Examples of such feedback may include, for example, suggestions such as “Increase leg drive” while executing a bench press, or “Maintain upright back posture” while executing a squat. Such feedback serves to improve specific movement patterns to better match the commonly accepted norms of good form.

In a second feedback loop 222, processed information is fed to the coach or trainer (which may be the user himself/herself), enabling the human in the loop to form another layer of feedback via mid-level feedback. By using the performance metrics provided as well as prior knowledge as an expert in the field, the trainer or coach may adapt the training program accordingly acting as another controller acting on the user/plant.

In a third feedback loop 224, feedback is achieved by combining the original workout program with the outputs from the first feedback loop 220 and the second feedback loop 222 to allow for automated processing of improvements to the training plan. The third feedback loop 224 uses aggregate data trends from the user base and trainer suggestions to ascertain which changes to the current program will maximize training efficiency and improve performance for the individual user.

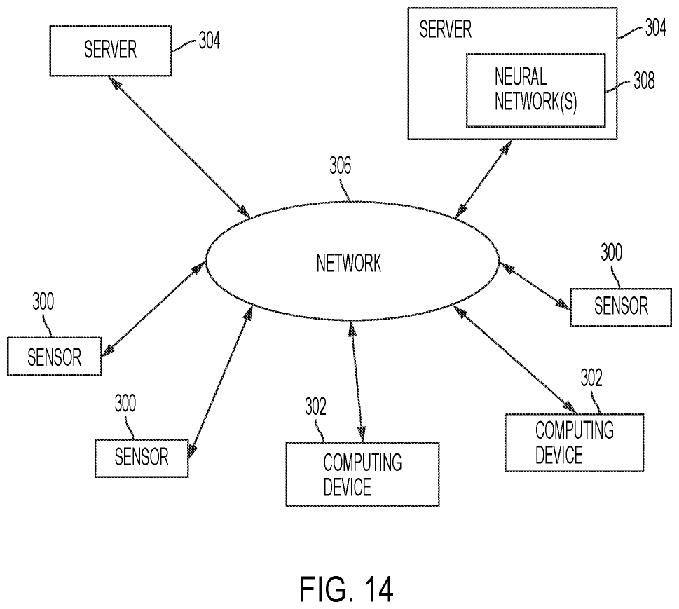

FIG. 14 is a diagram illustrating a general configuration of devices that communicate through a network, according to an embodiment.

Referring now to FIG. 14, one or more sensors (for example, sensors such as sensor 20 in FIG. 1) 300, one or more computing devices (for example, a smartphone, tablet, laptop, wearable computing device or desktop computer) 302 and one or more servers 304 are connected over a network 306. Through this configuration, the one or more sensors 300, the one or more computing devices 302 and the one or more servers 304 may be able to communicate with each other. Moreover, the network 306 is not limited to any network configuration and can include, for example, one or a combination of a local area network(s), a wide area network(s), the Internet, etc. Moreover, the embodiments are not limited to any particular communications protocols.

Further, the embodiments are not limited to any particular number of sensors 300, computing devices 302 and/or servers 304. As an example, a plurality of sensors 300 may be used to collect data from a plurality of user, and transmit the collected data over the network 306 to one or more servers 304.

Various embodiments disclosed herein use one or more neural networks, such as neural network(s) 308 shown in FIG. 14. The embodiments are not limited to a neural network being at any particular location. For example, a neural network can be located on a server or on a computing device shown in FIG. 14. Embodiments can include multiple neural networks which are on different devices. For example, one neural network may be on a server shown in FIG. 14, and another neural network may be on a computing device shown in FIG. 14.

Various computer programs described herein may be installed and/or running on the server(s), the computing device(s), and/or the sensor(s). However, the embodiments are not limited to any particular distribution of hardware/software, or to the specific configuration shown in the figures. Instead, there are many different configurations and many different manners in which hardware/software can be distributed to implement various embodiments.

Various embodiments can be implemented by a processor or processors running software (computer-readable instructions) stored in memory. Such processors may include, for example, one or more central processing units (CPUs) and/or one or more graphics processing units (GPUs). However, embodiments are not limited to any particular type of processors.

Program(s)/software implementing embodiments disclosed herein may be recorded on a non-transitory computer-readable media. Examples of a non-transitory computer-readable media include a magnetic recording apparatus, an optical disk, a magneto-optical disk, and/or volatile and/or non-volatile semiconductor memory (for example, RAM, ROM, etc.). Examples of the magnetic recording apparatus include a hard disk device (HDD), a flexible disk (FD), and a magnetic tape (MT). Examples of the optical disk include a DVD (Digital Versatile Disc), DVD-ROM, DVD-RAM (DVD-Random Access Memory), BD (Blue-ray Disk), a CD-ROM (Compact Disc-Read Only Memory), and a CD-R (Recordable)/RW.

As described herein, various embodiments may be implemented to track human or object motion by applying data filtering, artificial intelligence, and data analytics to process data generated by a sensor coupled to a barbell.

Embodiments may be used for purposes including, but not limited to (a) tracking physical training and performance, (b) identifying and quantifying activities based on movements, (c) identifying individuals based on movements, (d) identifying deviations from planned activities and motions, (e) predicting performance by mapping captured data to quantitative metrics, (f) communicating performance through unique metrics, (g) identifying or predicting performance setbacks such as plateaus, deficiencies, or risks of injuries, (h) deriving suggestions for variations to movement and/or activity to enhance performance and/or decrease risks, and (i) creating training aids, which use processed data to enhance activity experience by promoting safety, efficiency, enjoyment, and/or advancement.

Applications for embodiments discloses herein include, but are not limited to, (a) barbell exercises including, but not limited to Olympic lifts (i.e., snatch, clean, and jerk variants), power lifts (i.e., squat, bench, and deadlift variants), other barbell lifts (i.e., rows, push press, and overhead press), (b) dumbbell, kettlebell, and other free-weight exercises, (c) other weight room and physical therapy activities, including, but not limited to, lifting machines using cables or lever arms, body weight exercises, power sleds, battle ropes, (d) football blocking and tackling sleds, including power output/rate of force production (explosiveness), (e) swinging motions in activities such as baseball, softball, golf, and tennis, (f) throwing motions and object flight in activities such as baseball, softball, football, and darts, (g) pose recognition and posture and stability monitoring in activities such as stretching, yoga, and gymnastics, (h) gait analysis, (i) biking (i.e., pedaling rhythm analysis, suggestions of gear ratios), (j) winter sports (skiing, snowboarding, snowmobiling, etc.), (k) motion analytics during turns to optimize speed during racing, (I) rotation analysis for posture or tricks.

Although embodiments have been described herein in detail, it should be understood that the various changes, substitutions, and alterations could be made hereto without departing from the spirit and scope of the embodiments.

What is claimed is:

1. An apparatus comprising:
at least one neural network configured to:
receive data generated by a sensor coupled to a barbell, indicating at least one of acceleration of the barbell over time, angular velocity of the barbell over time, and magnetic field effects due to movement of the barbell over time, and labeled to indicate a type of exercise performed with the barbell and an identity of a user that performed the exercise with the barbell, and
detect repetitions of the exercise performed with the barbell from the received data.

2. An apparatus as in claim 1, wherein the at least one neural network is further configured to count the repetitions.

3. An apparatus as in claim 1, further comprising:
at least one other neural network configured to, prior to the data generated by the sensor being received by the at least one neural network, detect the type of exercise performed with the barbell from the data generated by the sensor, and label the data generated by the sensor to indicate the type of exercise performed with the barbell.

4. An apparatus as in claim 1, further comprising:
at least one additional neural network configured to, prior to the data generated by the sensor being received by the at least one neural network, detect the identity of the user that performed the exercise with the barbell from the data generated by the sensor, and label the data generated by the sensor to indicate the identity of the user that performed the exercise with the barbell.

5. An apparatus as in claim 1, further comprising:
at least one other neural network configured to, prior to the data generated by the sensor being received by the at least one neural network, detect one of the type of exercise performed with the barbell and the identity of the user that performed the exercise with the barbell from the data generated by the sensor, and label the data generated by the sensor to indicate the one of the type of exercise performed with the barbell and the identity of the user that performed the exercise with the barbell; and
at least one additional network configured to, prior to the data generated by the sensor being received by the at least one neural network, detect the other of the type of exercise performed with the barbell and the identity of the user that performed the exercise with the barbell, from the data generated by the sensor and labeled to indicate the one of the type of exercise performed with the barbell and the identity of the user that performed the exercise with the barbell, and label the data generated by the sensor to indicate the other of the type of exercise performed with the barbell and the identity of the user that performed the exercise with the barbell.

6. An apparatus as in claim 1, further comprising:
at least one additional neural network configured to detect a bounded set from the data generated by the sensor, prior to the data generated by the sensor being labeled to indicate the type of exercise performed with the barbell and the identity of a user that performed the exercise with the barbell,
wherein the data generated by the sensor received by the at least one neural network is data of the detected bounded set having been labeled to indicate the type of exercise performed with the barbell and the identity of a user that performed the exercise with the barbell.

7. An apparatus comprising:
at least one memory storing instructions; and
at least one processor that executes the instructions to perform a process including:
receiving data generated by a sensor coupled to a barbell, and indicating at least one of acceleration of the barbell over time, angular velocity of the barbell over time, and magnetic field effects due to movement of the barbell over time,
detecting, from the received data, a type of exercise performed with the barbell and an identity of a user that performed the exercise with the barbell, and
using the detected type of the exercise performed with the barbell and the detected identity of the user that performed the exercise with the barbell to detect, from the received data, repetitions of the exercise performed with the barbell.

8. The apparatus as in claim 7, wherein the using the detected type of the exercise performed with the barbell and the detected identity of the user that performed the exercise with the barbell to detect, from the received data, repetitions of the exercise performed with the barbell, comprises:
passing the received data, information indicating the detected type of exercise performed with the barbell, and information indicating the detected identity of the user that performed the exercise with the barbell, through at least one trained neural network, to:
determine, for each repetition of the exercise performed with the barbell by the user, when the repetition occurs, to thereby detect each repetition.

9. The apparatus in claim 8, wherein the at least one trained neural network includes a trained convolutional neural network and/or a trained recurrent neural network.

10. An apparatus as in claim 8 wherein the process further comprises:
counting each detected repetition.

11. The apparatus as in claim 7, wherein the using the detected type of the exercise performed with the barbell and the detected identity of the user that performed the exercise with the barbell to detect, from the received data, repetitions of the exercise performed with the barbell, comprises:
passing the received data, information indicating the detected type of exercise performed with the barbell, and information indicating the detected identity of the user that performed the exercise with the barbell, through at least one trained neural network, to detect and count the repetitions.

12. The apparatus in claim 11, wherein the at least one trained neural network includes a trained convolutional neural network and/or a trained recurrent neural network.

13. The apparatus of claim 7, wherein the detecting the type of exercise performed with the barbell and the identity of a user that performed the exercise with the barbell comprises:
passing the received data through at least one trained neural network to detect the type of exercise performed with the barbell from the received data.

14. The apparatus of claim 7, wherein the detecting the type of exercise performed with the barbell and the identity of a user that performed the exercise with the barbell comprises:
passing the received data through at least one trained neural network to detect the identity of the user that performed the exercise with the barbell from the received data.

15. The apparatus of claim 7, wherein the detecting the type of exercise performed with the barbell and the identity of a user that performed the exercise with the barbell comprises:
passing the received data through at least one trained neural network to detect, from the received data, the type of exercise performed with the barbell and the identity of the user that performed the exercise with the barbell.

16. The apparatus as in claim 7, wherein the detecting the type of exercise performed with the barbell and the identity of a user that performed the exercise with the barbell comprises:
passing the received data through a first trained neural network to detect one of the type of exercise performed with the barbell and the identity of the user that performed the exercise with the barbell, and
passing the received data and information indicating the detected one of the type of exercise performed with the barbell and the identity of the user that performed the exercise with the barbell, through a second trained neural network to detect the other of the type of exercise performed with the barbell and the identity of the user that performed the exercise with the barbell.

17. An apparatus comprising:
at least one memory storing instructions; and
at least one processor that executes the instructions to perform a process including:
receiving data generated by a sensor coupled to a barbell, and indicating at least one of acceleration of the barbell over time, angular velocity of the barbell over time, and magnetic field effects due to movement of the barbell over time,
passing the received data through at least one trained neural network to detect, from the received data, a type of exercise performed with the barbell and an identity of a user that performed the exercise with the barbell, and
passing the received data, labeled by the detected type of exercise performed with the barbell and the detected identity of the user that performed the exercise with the barbell, though at least one additional trained neural network to detect, from the labeled data, repetitions of the exercise performed with the barbell.

18. The apparatus as in claim 17, wherein the passing the received data through at least one trained neural network to detect, from the received data, the type of exercise performed with the barbell and the identity of a user that performed the exercise with the barbell, comprises:
passing the received data through a first trained neural network to detect one of the type of exercise performed with the barbell and the identity of the user that performed the exercise with the barbell, and
passing the received data and information indicating the detected one of the type of exercise performed with the barbell and the identity of the user that performed the exercise with the barbell, through a second trained neural network to detect the other of the type of exercise performed with the barbell and the identity of the user that performed the exercise with the barbell.

19. An apparatus as in claim 17, wherein the passing the received data, labeled by the detected type of exercise performed with the barbell and the detected identity of the user that performed the exercise with the barbell, though at least one additional trained neural network, counts the repetitions of the exercise performed with the barbell.

20. An apparatus as in claim 17, wherein the process further comprises:
counting the detected repetitions.

21. An apparatus as in claim 17, further comprising:
passing the data generated by the sensor through least one other neural network to detect a bounded set from the data generated by the sensor, prior to the data being labeled to indicate the type of exercise performed with the barbell and the identity of a user that performed the exercise with the barbell,
wherein the data passed through the at least one additional trained neural network is data of the detected bounded set.

22. An apparatus comprising:
at least one memory storing instructions; and
at least one processor that executes the instructions to perform a process including:
receiving data generated by a sensor coupled to a barbell, and indicating at least one of acceleration of the barbell over time, angular velocity of the barbell over time, and magnetic field effects due to movement of the barbell over time, and
using information indicating an identity of a user that performed an exercise with the barbell and information indicating a type of the exercise, to detect, from the received data, repetitions of an exercise performed with the barbell.

23. The apparatus as in claim 22 wherein the using information indicating the identity of a user that performed the exercise with the barbell and information indicating the type of the exercise, to detect, from the received data, repetitions of an exercise performed with the barbell, comprises:
passing the received data, the information indicating the identity of the user that performed the exercise with the barbell, and the information indicating the type of the exercise through at least one trained neural network to detect the repetitions of the exercise performed with the barbell.

24. The apparatus in claim 23, wherein the at least one trained neural network includes a trained convolutional neural network and/or a trained recurrent neural network.

25. The apparatus as in claim 22, wherein the using information indicating the identity of a user that performed the exercise with the barbell and information indicating the type of the exercise, to detect, from the received data, repetitions of an exercise performed with the barbell, comprises:
passing the received data, the information indicating the identity of the user that performed the exercise with the barbell, and the information indicating the type of the exercise through at least one trained neural network to detect and count the repetitions.

26. The apparatus in claim 25, wherein the at least one trained neural network includes a trained convolutional neural network and/or a trained recurrent neural network.

27. The apparatus as in claim 23, further comprising:
obtaining the information indicating the identity of the user that performed the exercise with the barbell and the information indicating the type of the exercise as labeled data from a computer program.

28. The apparatus as in claim 22 wherein the using information indicating the identity of a user that performed the exercise with the barbell and information indicating the type of the exercise, to detect, from the received data, repetitions of an exercise performed with the barbell, comprises:
labeling the received data with the identity of the user and the type of exercise, and
passing the labeled data through at least one trained neural network to detect the repetitions of the exercise performed with the barbell.

29. The apparatus as in claim 22 wherein the using information indicating the identity of a user that performed the exercise with the barbell and information indicating the type of the exercise, to detect, from the received data, repetitions of an exercise performed with the barbell, comprises:
labeling the received data with the identity of the user and the type of exercise, and
passing the labeled data through at least one trained neural network to detect and count the repetitions of the exercise performed with the barbell.
